(19) United States
(12) Patent Application Publication
(10) Pub. No.: US 2026/0338753 A1
(43) Pub. Date: Oct. 29, 2026

(54) PREDICTION AND EARLY WARNING SYSTEM FOR WATER QUALITY AT MONITORING-SCARCE POINT BASED ON LIQUID GRAPH NEURAL NETWORK

(71) Applicant: Harbin Institute of Technology, Harbin (CN)

(72) Inventors: Yu TIAN, Harbin (CN); Jiarong LI, Harbin (CN); Lipin LI, Harbin (CN)

(21) Appl. No.: 19/656,556

(22) Filed: Apr. 23, 2026

(30) Foreign Application Priority Data
Apr. 24, 2025 (CN) 202510518993X

(51) Int. Cl. G01N 33/18; G06N 3/042; G06N 3/08

(52) U.S. Cl. CPC G01N 33/18; G06N 3/042; G06N 3/08

(57) ABSTRACT

A system for the water quality based on a liquid graph neural network includes watershed front-end monitoring devices, an edge computing apparatus and a central server; the apparatus constructs a directed initial watershed graph, historical flow velocities, a riverbed permeability coefficient and hydrological connectivity data, and dynamically corrects edge weights based on real-time flow velocity gradients and rainfall changes; and generates a dynamic adjacency matrix and outputs predicted values for water quality parameters and alarm levels within a future preset time domain at a data-scarce point by using the neural network; when the predicted value exceeds a preset threshold, the apparatus sends a hardware action instruction to an associated upstream node or downstream node to control an automatic sampler to perform encrypted sampling, and calls a local model and locally cached data to maintain continuous early warning when a response delay of the central server reaches a preset condition.

### CROSS-REFERENCE TO RELATED APPLICATION

This application claims priority from the Chinese patent application 202510518993X filed Apr. 24, 2025, the content of which is incorporated herein in the entirety by reference.

### TECHNICAL FIELD

The present invention relates to the technical field of water environment monitoring and intelligent early warning, and more particularly, to a prediction and early warning system for the water quality at an edge side based on a liquid graph neural network and oriented to data-scarce points.

### BACKGROUND ART

In the existing watershed monitoring system, the conventional hydrological monitoring device can achieve high-density spatial grid layout because of its simple structure, high stability and no maintenance. However, due to the constraints of construction cost, operation & maintenance capabilities, geographical accessibility and other factors, water quality monitoring devices are generally only sparsely distributed in a main stream or key section, resulting in a large number of tributaries and monitoring blind areas around potential pollution sources. Therefore, how to use the existing hydrological conditions and limited water quality devices to expand water quality perception and early warning capabilities at data-scarce points has become the actual demand in the fine supervision of a watershed.

With the deep penetration of artificial intelligence into the field of water environments, data-driven modeling provides a new and reliable tool for early warning of watershed water quality. However, under the condition of discontinuous or insufficient coverage of monitoring data, the existing machine learning algorithms rely on historical data under static topological constraints for reasoning, which can easily cause errors to propagate and accumulate between nodes and restrict the robustness and generalization performance of the model. In view of data-scarce points, multi-point risk deduction and continuous operation under abnormal communication conditions need to be achieved at the same time. The prior art still has shortcomings.

### SUMMARY

The problem to be solved by the present invention is as follows: in a watershed scenario where water quality monitoring devices are sparsely distributed, how to achieve continuous, accurate and executable water quality prediction and early warning for data-scarce points without water quality sensors under the conditions of meeting downstream propagation constraints, time causal constraints and edge-side real-time control requirements.

In order to solve the above technical problem, the present invention provides a prediction and early warning system for the water quality at a monitoring-scarce point based on a liquid graph neural network, including watershed front-end monitoring devices, an edge computing apparatus and a central server, wherein the edge computing apparatus calls a directed initial watershed graph, an initial adjacency matrix, a degree matrix and a feature matrix to perform water quality parameter prediction, alarm level determination and hardware action control for data-scarce points.

The watershed front-end monitoring devices are deployed at a plurality of monitoring nodes; the monitoring nodes include observation nodes deployed with water quality sensors and data-scarce points where no water quality sensor is deployed; each watershed front-end monitoring device includes at least a hydrological sensor and a communication module, and the watershed front-end monitoring device deployed at an observation node further includes a water quality sensor and an automatic sampler and is configured to collect real-time hydrological data of each monitoring node, collect real-time water quality data of each observation node and execute a hardware action instruction. The watershed front-end monitoring device includes a water quality sensor, a hydrological sensor and an embedded control unit and is configured to receive a control instruction from the edge computing apparatus and execute corresponding physical operations.

The edge computing apparatus is deployed in a watershed monitoring network and in communication connection with the watershed front-end monitoring devices; the edge computing apparatus includes a memory and at least one processor, the memory stores a liquid graph neural network model, a directed initial watershed graph, an initial adjacency matrix, a degree matrix, a feature matrix, a physical constraint rule and a computer program therein; and the processor, when executing the computer program, is configured to perform error value deletion, threshold capping, missing value filling, normalization and time step alignment processing on the real-time hydrological data and the real-time water quality data; construct or call a directed initial watershed graph and generate a dynamic connection weight according to a riverway distance, historical flow velocities, a riverbed permeability coefficient and hydrological connectivity data, generate a dynamic adjacency matrix by using hidden state update driven by a liquid differential equation, a topological channel update network and a hydrological channel update network as well as a joint attention fused with a space mask matrix and a time mask matrix, and accordingly output predicted values for water quality parameters and corresponding alarm levels at a plurality of data-scarce points within a future preset time domain; when the predicted value for the water quality parameter exceeds a preset threshold or the alarm level determined according to the predicted values for the water quality parameters reaches a preset level, generate an alarm data packet and send a hardware action instruction to an upstream node or a downstream node associated with the data-scarce point, to control its automatic sampler to perform encrypted sampling. The edge computing apparatus includes an industrial processor, a memory, a communication interface module and an I/O control module connected to a field sensor and an actuator, wherein the processor is configured to perform continuous time solving of the liquid graph neural network model; the communication interface module includes a 4G/5G or LoRa communication unit; and the I/O control module is configured to output control signals to an automatic sampler, an electromagnetic valve or an alarm apparatus.

The central server is in communication connection with the edge computing apparatus and configured to receive prediction results, alarm records, device operation state information and model operation logs uploaded by an edge side, and issues a model update strategy, model parameters or an incremental update packet to the edge computing apparatus; and when a network is interrupted or a response delay of the central server reaches a preset condition, the edge computing apparatus calls locally cached real-time hydrological data and a local model to independently maintain prediction and early warning, alarm level determination and local alarm output for the data-scarce points.

In some embodiments, the edge computing apparatus is configured to construct a watershed graph structure for the observation nodes and the data-scarce points. Specifically, the edge computing apparatus acquires riverway data, historical hydrological data and hydrological connectivity data of each node; determines the initial connection weight between each pair of adjacent nodes according to a riverway distance, historical flow velocities and a riverbed permeability coefficient between each pair of adjacent nodes having a downstream relationship, and establishes directed edges only when there is a downstream relationship and hydrological connection; constructs the initial adjacency matrix and the degree matrix according to the riverway distance, an average node distance and a distance attenuation coefficient; and constructs the feature matrix of each node according to the historical hydrological data and the hydrological connectivity data of the node and historical water quality data of the observation nodes, wherein the water quality characterization features at the data-scarce points are estimated according to the historical water quality data of adjacent observation nodes.

In some embodiments, the edge computing apparatus is configured to dynamically update a dynamic connection weight of the watershed graph structure based on an inter-node downstream topological relationship and real-time hydrological perturbations. Specifically, a dynamic correction factor between each pair of adjacent nodes is determined according to the initial connection weight between each pair of adjacent nodes and the real-time hydrological data of each node, and the dynamic connection weight between each pair of adjacent nodes is determined according to the initial connection weight and the dynamic correction factor, to reflect dynamic effects of real-time hydrological conditions such as flow velocity gradients and rainfall changes on a water quality transport relationship between the nodes.

In some embodiments, the edge computing apparatus is configured to perform continuous time state update through a preset liquid graph neural network model according to a dynamically updated graph structure to obtain a hidden state of each node and dynamically adjust an adjacency matrix of the graph structure. Specifically, the hidden state update in the liquid graph neural network model is driven by a preset liquid differential equation; the edge computing apparatus determines a feature vector in the feature matrix of each node, and generates a comprehensive input according to the feature vector, the dynamic connection weight and a set of nodes adjacent to this node to output the hidden state of each node, thereby achieving a dynamic evolution of the node state under continuous time conditions.

Further, in some embodiments, the edge computing apparatus determines an attention score based on the hidden state at the previous moment, and act the space mask matrix and the time mask matrix to the attention score to obtain a joint attention; and constructs a topological channel update network and a hydrological channel update network according to a graph convolutional network, updates the initial adjacency matrix, and fuses the joint attention to obtain the dynamic adjacent matrix of each node. The space mask matrix is used to mask a countercurrent relationship; the time mask matrix is used to mask future moment data and data that exceeds a permitted upload delay, thereby restraining invalid information transmission and future information interference on the basis of satisfying downstream propagation constraints and time causal constraints; and the dynamic adjacent matrix is further used to determine an upstream node or a downstream node associated with the data-scarce point that reaches the alarm condition, to control its automatic sampler to perform encrypted sampling.

In some embodiments, the central server is configured to perform training, verification, version update and issuance on the liquid graph neural network model. Specifically, the central server acquires training samples constructed based on the historical hydrological data, the historical water quality data and the hydrological connectivity data; trains and verifies the liquid graph neural network model according to the training samples to obtain an updated liquid graph neural network model; when hydrological seasonal features of the watershed change or monitoring node data is newly added, re-optimizes model parameters to generate an incremental update packet; and when a network connection state meets a preset stable condition, issues a model update instruction and the incremental update packet to the edge computing apparatus to achieve edge-side model version update.

In some embodiments, the edge computing apparatus is configured to call the liquid graph neural network model issued by the central server, perform local reasoning on the plurality of data-scarce points at the edge side, and output predicted values for water quality parameters and corresponding alarm levels at the plurality of data-scarce points within a future preset time domain; and when the predicted value for the water quality parameter exceeds a preset threshold or the alarm level reaches a preset level, generate alarm event information and send the alarm event information to the central server, and meanwhile send the hardware action instruction to upstream nodes or downstream nodes associated with the data-scarce points, to control their automatic samplers to perform encrypted sampling.

In some embodiments, the network being interrupted or the response delay of the central server reaching the preset condition includes at least one of interruption of a communication link between the edge computing apparatus and the central server, a packet loss rate exceeding a preset threshold, and a communication delay exceeding a preset delay threshold; and when the network is interrupted or the response delay of the central server reaches the preset condition, the edge computing apparatus calls a locally stored liquid graph neural network model version and locally cached real-time hydrological data, and continues to execute edge reasoning and early warning output for the plurality of data-scarce points, thereby ensuring the continuous operation in the case of abnormal communication, insufficient response at a center side or emergency scenarios, and improving the emergency early warning reliability and operation robustness of the system.

In some embodiments, the edge computing apparatus and the central server operate in an edge-cloud collaboration mode; the edge computing apparatus uploads the prediction results, alarm records, device operation state information and model operation logs at the data-scarce points to the central server; the central server executes model evaluation, policy adjustment and version synchronization according to information uploaded by the edge side, and generates a model update strategy, model parameters or an incremental update packet related to prediction and early warning for the data-scarce points; and the edge computing apparatus updates an edge-side reasoning process according to the model update strategy, the model parameters or the incremental update packet issued by the central server, so as to complete edge-side model collaboration update under the condition of not interrupting the prediction and early warning output at the data-scarce points.

The present invention provides a prediction and early warning system for the water quality at a monitoring-scarce point based on a liquid graph neural network, which at least has the following beneficial effects.

The watershed front-end monitoring devices, the edge computing apparatus and the central server constitute a deployable watershed water quality prediction and early warning system architecture, which can meet application requirements for real-time prediction, early warning push and automatic sampling disposal at the monitoring-scarce point in a watershed scenario.

By constructing the directed initial watershed graph on the edge computing apparatus side, and using observation node data of a small number of deployed water quality monitoring devices combined with widely collected real-time hydrological data, the water quality deduction capability is formed at the data-scarce points, and the risk deduction coverage of more points is supported by less high-cost water quality devices, thereby greatly reducing the overall layout cost of the system.

By introducing the topological channel update network, the hydrological channel update network and the joint attention mechanism with embedded physical constraints in the process of generating the dynamic adjacency matrix, invalid information transmission and future information interference can be suppressed on the basis of satisfying the downstream propagation constraints and time causal constraints, and a hysteresis effect and path dependence of pollutants can be more accurately captured to ensure that the prediction results more conform to actual migration laws of pollutants.

By setting an edge-cloud collaboration mechanism between the central server and the edge computing apparatus, the collaboration management of model training, verification, incremental update and edge deployment is achieved. At the same time, in case of network interruption or response delay of the central server, continuous prediction and early warning and local alarm output can still be maintained through the local model and locally cached data, which significantly enhances the continuous operation capability and emergency decision-making support capability of the system under complex working conditions and abnormal communication conditions.

### BRIEF DESCRIPTION OF THE DRAWINGS

FIG. 1 shows an overall architecture diagram of a system in an embodiment of the present invention;

FIG. 2 shows a schematic flowchart of prediction and early warning for the water quality at data-scarce points in an embodiment of the present invention;

FIG. 3 shows a schematic diagram of watershed scenario deployment and graph structure construction in an example of the present invention;

FIG. 4 shows a schematic diagram of a dynamic coding mechanism for spatiotemporal features driven by liquid neurons in an example of the present invention;

FIG. 5 shows a schematic diagram of a liquid causality perception mechanism in an embodiment of the present invention;

FIG. 6 shows a schematic diagram of training, updating and issuing processes of a central server in an embodiment of the present invention;

FIG. 7 shows a schematic diagram of edge reasoning and local disaster tolerance in an embodiment of the present invention;

FIG. 8 shows a comparison diagram of predicted NSE effects of different models in an embodiment of the present invention; and

FIG. 9 shows a comparison diagram between prediction and actual measurement of total nitrogen in water quality parameters in an embodiment of the present invention.

### DETAILED DESCRIPTION OF THE DISCLOSURE

In order to make the above objects, features and advantages of the present invention clearer, the specific embodiments of the present invention are described in detail in conjunction with the accompanying drawings.

It should be noted that, relation terms such as “first” and “second” in the present invention are used merely to distinguish a subject or an operation from another subject or another operation, and not to necessarily require or imply any substantial relation or sequence between these subjects or operations. Moreover, terms “comprise/include”, “contain” or any variation thereof are intended to cover a nonexclusive containing, such that a process, a method, an item or a device containing a series of elements not only includes these elements, but also includes other elements that are not set forth specifically, or also includes an inherent element of such a process, method, item or device. Without further limitation, an element defined by a phrase “include a . . . ” does not mean that other elements are excluded from the process, method, item or device including the same element.

In the description of the present specification, the description of referring terms such as “embodiment”, “an embodiment” and “an implementation” means that particular features, structures, materials or characteristics described in combination of the embodiment or implementation are included in at least one embodiment or exemplary implementation of the present invention. In the present specification, schematic description of the above terms does not necessarily refer to the same embodiment or implementation. Furthermore, the described particular features, structures, materials or characteristics can be integrated in any one or more embodiments or implementations in a proper manner.

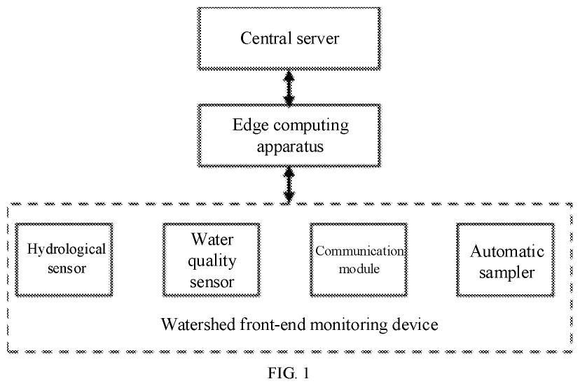

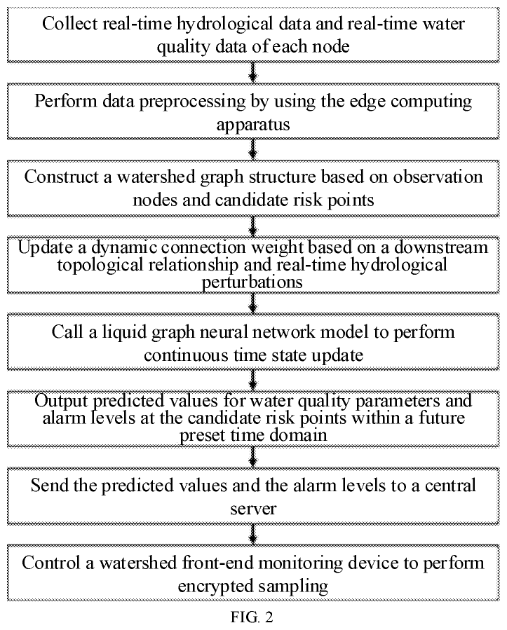

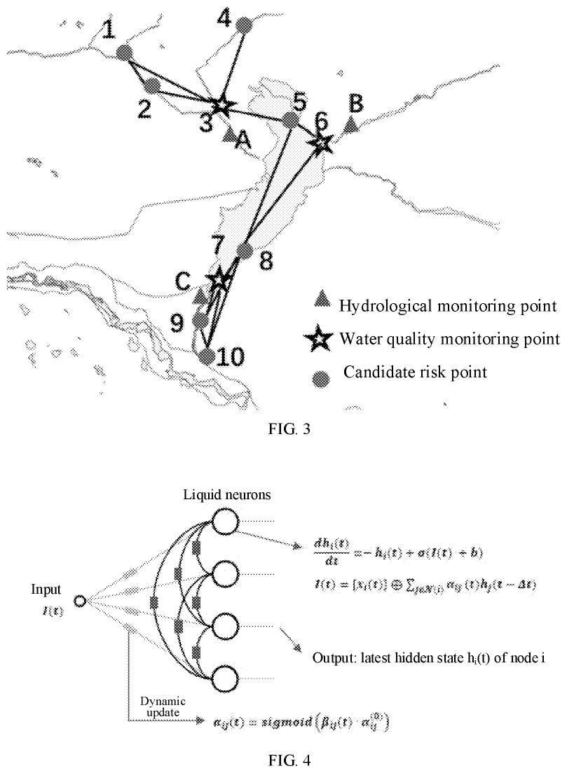

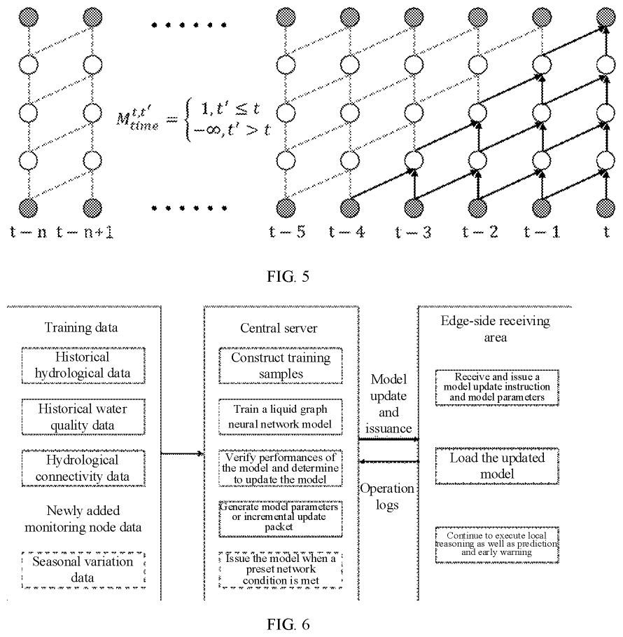

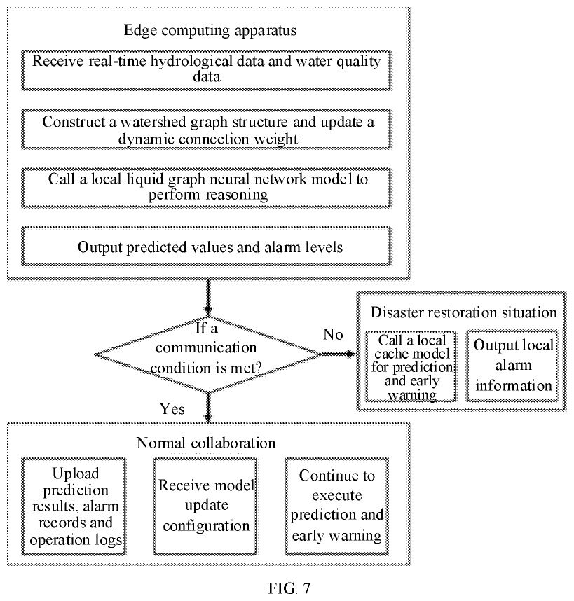

In one embodiment, referring to FIG. 1, FIG. 2, FIG. 3, FIG. 6 and FIG. 7, the present invention provides a prediction and early warning system for the water quality at a monitoring-scarce point based on a liquid graph neural network. The system is composed of watershed front-end monitoring devices, an edge computing apparatus and a central server. The edge computing apparatus is located at a watershed side and directly receives data from monitoring nodes, and is configured to perform water quality parameter prediction, alarm level determination and hardware action control for data-scarce points based on a directed initial watershed graph, a dynamic connection weight, a hidden state update driven by a liquid differential equation and a dynamic adjacency matrix generation process. The central server is in communication connection with the edge computing apparatus and configured to complete training, verification, version update and issuance of the liquid graph neural network model to support the continuous optimization and online operation of an edge-side model.

As shown in FIG. 3, in the present embodiment, points numbered 1, 2, 4, 5, 8, 9 and 10 are data-scarce points, points numbered 3, 6 and 7 are observation nodes of deployed water quality monitoring devices, and A, B and C represent hydrological monitoring points. For any two nodes, only if there is a downstream relationship and hydrological connection between the nodes, they are defined as adjacent nodes, and directed edges are established in a graph structure to reflect a downstream propagation relationship of pollutants along a riverway in a watershed.

In some application scenarios, when the edge computing apparatus detects that a network between the edge computing apparatus and the central server is interrupted or a response delay reaches a preset condition, the edge computing apparatus calls locally cached data and a local model to continue to maintain prediction and early warning, alarm level determination and local alarm information output for the data-scarce points. Therefore, the system of the present invention not only can reduce the burden of new arrangement and operation & maintenance of high-cost water quality monitoring devices, but also can adapt to continuous operation requirements under a complex field environment.

As an optional embodiment of the present invention, as shown in FIG. 3, the edge computing apparatus receives real-time hydrological data and real-time water quality data from the observation nodes, and constructs a watershed graph structure for the observation nodes and the data-scarce points. Nodes in the watershed graph structure include two types: one type is observation nodes that have been deployed with water quality monitoring devices and can provide historical water quality data and real-time water quality data; and the other type is data-scarce points that have not been deployed with water quality monitoring devices but need prediction and early warning for the water quality. For any two nodes, only if there is a downstream relationship and hydrological connection between the nodes, they are defined as adjacent nodes, and directed edges are established in the graph structure to reflect a downstream propagation relationship of pollutants along a riverway in a watershed.

In the present embodiment, in the stage of system deployment, the riverway data, historical hydrological data and hydrological connectivity data of each monitoring node are acquired in advance, and a directed initial watershed graph is constructed based on the above data and then stored in the edge computing apparatus. The riverway data at least includes a riverway distance, a riverway flow velocity, a riverbed permeability coefficient, an average node distance and a distance attenuation coefficient; the historical hydrological data at least includes historical meteorological time series data, historical water quality data and vegetation coverage indexes; and the hydrological connection data at least includes a spatial topological relationship of the nodes. Based on the above data, the initial connection weight between each pair of adjacent nodes having a downstream relationship may be determined, and the initial adjacency matrix, a degree matrix and a feature matrix are further constructed.

In the stage of system operation, the edge computing apparatus directly calls the directed initial watershed graph pre-stored in the memory, and receives the real-time hydrological data and real-time water quality data uploaded by the observation nodes; and accordingly, dynamically corrects edge weights in the directed initial watershed graph according to flow velocity gradients and rainfall changes in the real-time hydrological data to generate a dynamic watershed graph which represents an actual transport relationship of pollutants at the current moment between the observation nodes and the data-scarce points.

As an optional embodiment of the present invention, the initial connection weight is determined by using an improved attenuation coefficient formula:

$${α}_{ij}^{0} = \frac{1}{1 + {e}^{0.5 ( {L}_{ij} - \frac{v}{k} )}} ;$$

in which, αᵢⱼ⁰ represents the initial connection weight; Lᵢⱼ represents a riverway distance from nodes i to j; i is any node; j is any node except i, and is cyclical, for example, when i=1; j is 2-10; when i=2, j is 1 and 3-10; v represents a river flow velocity; and k represents a riverbed permeability coefficient.

The initial adjacency matrix of each node is constructed according to the riverway distance between each pair of adjacent nodes, an average node distance of the nodes and a distance attenuation coefficient; and the degree matrix of each node is constructed according to the initial adjacency matrix of each node.

Specifically, the initial adjacency matrix is as follows:

$${A}_{ij}^{0} = \{ \begin{matrix}\frac{1}{1 + {( \frac{{L}_{ij}}{{L}_{0}} )}^{α}} , & node j is located upstream of \\ & i and is hydrologically connected \\ 0 , & others\end{matrix} ;$$

in which, Aᵢⱼ⁰ represents the initial adjacency matrix; Lᵢⱼ represents a riverway distance from nodes i to j; L₀ represents an average distance between each pair of adjacent nodes in the watershed; and a represents a distance attenuation index, taking 1.5-2.0, which reflects the diffusion intensity of pollutants over distance. For the determination of L₀, for example, under the premise of a connection relationship, for example, Lᵢⱼ is 1 km, 5 km and 10 km respectively, L₀=(1+5+10)/3=5.33.

The feature matrix X contains the attributes of each node, such as water quality parameters, meteorological factors, etc. The attributes of each node have 22 dimensions, among which static attributes, dynamic time series, spatial topology and other multi-dimensional features are integrated. Details are given in Table 1 below.

TABLE 1

| | | | Output |
|---|---|---|---|
| Feature type | Original parameters | Encoding mode | dimension |
| Static | Elevation and land use type | Direct assignment | 2 |
| properties | | | |
| Dynamic | Total nitrogen, ammonia | Direct | 7 × 2 = 14 |
| time series | nitrogen, DO, rainfall capacity, | assignment, | |
| | maximum temperature, runoff | average of the | |
| | volume, and vegetation cover | previous 7 days | |
| | index | | |
| Spatial | Upstream and downstream | Direct assignment | 3 |
| topology | relationships, number of adjacent | | |
| | nodes, and dynamic connection | | |
| | weight | | |
| Adversarial | Confidence, water quality | Direct assignment | 3 |
| generation | features of a generating node, and | | |
| | meteorological data of the | | |
| | generating node | | |

According to the adjacency matrix, the degree matrix is constructed. The degree matrix is a diagonal matrix, and its diagonal element is a sum of the number of connections of the corresponding node in the adjacency matrix.

An exemplary adjacency matrix is shown in Table 2.

TABLE 2

| | i | | | | | | | | | |
|---|---|---|---|---|---|---|---|---|---|---|
| j | 1 | 2 | 3 | 4 | 5 | 6 | 7 | 8 | 9 | 10 |
| 1 | 0.5 | 0 | 0 | 0 | 0 | 0 | 0 | 0 | 0 | 0 |
| 2 | 0 | 0.5 | 0 | 0 | 0 | 0 | 0 | 0 | 0 | 0 |
| 3 | 0 | 0.39 | 0.5 | 0.34 | 0 | 0 | 0 | 0 | 0 | 0 |
| 4 | 0 | 0 | 0 | 0.5 | 0 | 0 | 0 | 0 | 0 | 0 |
| 5 | 0 | 0 | 0.31 | 0 | 0.5 | 0.46 | 0 | 0 | 0 | 0 |
| 6 | 0 | 0 | 0 | 0 | 0.46 | 0.5 | 0 | 0 | 0 | 0 |
| 7 | 0 | 0 | 0 | 0 | 0.19 | 0.23 | 0.5 | 0 | 0 | 0 |
| 8 | 0 | 0 | 0 | 0 | 0 | 0 | 0.4 | 0.5 | 0 | 0 |
| 9 | 0 | 0 | 0 | 0 | 0 | 0 | 0.36 | 0.44 | 0.5 | 0 |
| 10 | 0 | 0 | 0 | 0 | 0 | 0 | 0.29 | 0.33 | 0.41 | 0.5 |

Exemplarily, the degree matrix obtained according to Table 2 is shown in Table 3.

TABLE 3

| | i | | | | | | | | | |
|---|---|---|---|---|---|---|---|---|---|---|
| j | 1 | 2 | 3 | 4 | 5 | 6 | 7 | 8 | 9 | 10 |
| 1 | 0.5 | 0 | 0 | 0 | 0 | 0 | 0 | 0 | 0 | 0 |
| 2 | 0 | 1.3 | 0 | 0 | 0 | 0 | 0 | 0 | 0 | 0 |
| 3 | 0 | 0 | 1.89 | 0 | 0 | 0 | 0 | 0 | 0 | 0 |
| 4 | 0 | 0 | 0 | 0.84 | 0 | 0 | 0 | 0 | 0 | 0 |
| 5 | 0 | 0 | 0 | 0 | 1.46 | 0 | 0 | 0 | 0 | 0 |
| 6 | 0 | 0 | 0 | 0 | 0 | 1.19 | 0 | 0 | 0 | 0 |
| 7 | 0 | 0 | 0 | 0 | 0 | 0 | 1.97 | 0 | 0 | 0 |
| 8 | 0 | 0 | 0 | 0 | 0 | 0 | 0 | 1.67 | 0 | 0 |
| 9 | 0 | 0 | 0 | 0 | 0 | 0 | 0 | 0 | 1.71 | 0 |
| 10 | 0 | 0 | 0 | 0 | 0 | 0 | 0 | 0 | 0 | 1.53 |

In practical applications in the present embodiment, the historical water quality data, vegetation coverage indexes, meteorological time series data, etc. of monitoring points within the watershed are collected. The above data are preprocessed, including: looking up and deleting wrong values of various types of data; setting reasonable upper and lower thresholds, and performing capping processing on data exceeding the thresholds; filling missing values using a random forest regression model; normalizing to eliminate dimensional differences; ensuring that time steps of data at each site are consistent, downscaling hourly scale data averagely to daily scale data, and generating a spatiotemporally-aligned model input dataset; collecting vegetation cover indexes and meteorological time series data of the data-scarce points for the above data preprocessing; and generating historical water quality auxiliary pseudo-node data using an adversarial network to form a spatiotemporal dataset covering the whole watershed, thereby forming the feature matrix. The generation of the adjacency matrix and the degree matrix can take into account the dynamic connection weights.

As shown in FIG. 2, as an optional embodiment of the present invention, according to the initial connection weight between each pair of adjacent nodes and real-time hydrological data of each node, the edge computing apparatus determines the dynamic connection weight between each pair of adjacent nodes, including:

determining a dynamic correction factor between each pair of adjacent nodes according to the real-time hydrological data of each node and learning parameters, wherein the dynamic correction factor is used to characterize a driving relationship of a linear combination of flow velocity gradients and rainfall changes on the initial connection weight.

Specifically, a dynamic adjustment correction factor is expressed as follows:

$${β}_{ij} ( t ) = tanh ( {w}_{v} \cdot \frac{{v}_{j} ( t ) - {v}_{i} ( t )}{{L}_{ij}} + {w}_{r} \cdot \frac{\partial r ( t )}{\partial t} ) .$$

in which:
βᵢⱼ(t): a dynamic correction factor of a node i and a node j at time t;
tan h: hyperbolic tangent function;
v(t): section flow rate, including vⱼ(t) and vᵢ(t);
r(t): rainfall intensity;
wᵥ, wᵣ: learnable parameters; and
Lᵢⱼ: riverway distance from nodes i to j.

The dynamic connection weight between each pair of adjacent nodes is determined according to the initial connection weight and the dynamic correction factor.

Specifically, the dynamic connection weight is expressed as the following formula:

$${α}_{ij} ( t ) = sigmoid ( {β}_{ij} ( t ) \cdot {α}_{ij}^{0} ) .$$

$${α}_{ij}^{0}$$

represents the initial connection weight; αᵢⱼ(t) represents the dynamic connection weight, which is a quantitative representation of the information transmission strength between nodes in the model; and the connection strength is a weight of information transmission between the nodes. Higher ay means that the node j contributes more to the node i, and the higher the reference value of the model.

In practical applications in the present embodiment, a dynamic adjustment factor drives the dynamic connection weight to change through a real-time hydrological response equation. Through the linear combination of flow velocity gradients and rainfall changes, mechanism-data hybrid drive of hydrodynamic principles and machine learning is implemented. When the dynamic correction factor increases, it indicates that the transport of pollutants is enhanced, and a forward gain of the correction factor is connected with the weight; and when the dynamic correction factor is reduced, it indicates that the transport of the pollutants is weakened, and the correction factor suppresses the connection weight, such that a dynamic watershed graph more conforms to real-time hydrological conditions.

As an optional embodiment of the present invention, after obtaining the dynamic connection weight, the edge computing apparatus inputs the feature vector in the feature matrix of each node and the updated connection relationship into the preset liquid graph neural network model to perform continuous time state update, and obtaining the hidden state of each node includes:
determining the feature vector of the feature matrix of each node; and according to the feature vector and the dynamic connection weight, generating the comprehensive input of each node in the preset liquid differential equation, so as to output the hidden state of each node, wherein
the preset liquid differential equation is:

$$\frac{{dh}_{i} ( t )}{dt} = - {h}_{i} ( t ) + σ ( I ( t ) + b ) ;$$

$$I ( t ) = {W}_{s} [ {x}_{i} ( t ) ] ⊕ {\sum }_{j ∈ 𝒩 ( i )} {α}_{ij} ( t ) {h}_{j} ( t - Δ t ) ;$$

in which, hᵢ(t) represents a hidden state of a node i at time t, which is used to map a node feature xᵢ(t) into a hidden space; σ represents a nonlinear activation function, i.e., an ReLU function; b represents a learnable bias term; Wₛ represents a learnable parameter matrix, which is automatically learned through model training without manual setting, and is used for the mapping of the feature vector of the node to the hidden space; [xᵢ(t)] represents the feature vector of the node i at time t; αᵢⱼ(t) represents the dynamic connection weight; ⊕ represents a feature splicing operation; Δt represents a time delay; I(t) represents the comprehensive input; j represents the node adjacent to the node i; and N(i) represents a set of the nodes adjacent to the node i.

Specifically, through the above-mentioned continuous time evolution mechanism, the edge computing apparatus jointly codes node's own attributes, historical effects of adjacent nodes and real-time hydrological drive into the hidden state, such that the hidden state not only represents the node's own temporal and spatial attributes, but also reflects associated effects of the upstream and downstream nodes in a watershed network. Therefore, the system can form an implicit state representation reflecting the spatial heterogeneity and temporal dynamics of the watershed at the edge side, which can be used for the subsequent update of the dynamic adjacency matrix and the prediction and early warning output at the data-scarce points.

In practical applications in the present embodiment, through a continuous time evolution mechanism of liquid neurons (reflected in a preset liquid differential equation), a spatial topology dependence and time series features can be dynamically fused. The real-time update process of the node state is simulated using a liquid differential equation. Original node attributes, the adjacency matrix, the degree matrix and other data are dynamically coded by a liquid time constant network to generate a hidden state reflecting the spatiotemporal features of the node, and finally a dynamically updated spatiotemporal feature matrix (that is, the hidden state of each node) is output.

As an optional embodiment of the present invention, after obtaining the hidden state of each node, the edge computing apparatus further dynamically adjusts an adjacency relationship in the graph structure to generate the dynamic adjacency matrix for edge reasoning and hardware action control, including:

based on the hidden state of each node at the previous moment, determining an attention score; constructing a spatial mask matrix and a time mask matrix; fusing the space mask matrix and the time mask matrix and then acting on the attention score to obtain the joint attention.

Specifically, the hidden state at the previous moment is selected. The attention score reflects the importance of different elements in an input sequence. In the attention mechanism, the query matrix, the key matrix and the value matrix are all obtained by linear transformation of the hidden state. Then, a dot product of query and key is scaled and softmaxed to obtain the attention score, and combined with a mask matrix, to obtain a new hidden state.

The hidden state of each node at the previous moment is captured through a graph convolutional network, and the topological channel update network is constructed in combination with the degree matrix; a spatiotemporal convolution of a real-time flow velocity field matrix and a turbulent feature matrix is captured through the graph convolutional network, and the hydrological channel update network is constructed, wherein the real-time flow velocity field matrix and the turbulent feature matrix are determined according to the real-time hydrological data.

Specifically, the dynamic update of the connection weight between the nodes is driven based on real-time hydrological data. An unsteady-state transport process of pollutants as hydrological conditions change is simulated. Through the spatiotemporal convolution of flow velocity field and turbulent features, local water flow sudden changes caused by heavy rain, gate control and other events are captured, and a hydrological channel network is constructed. A propagation mode of the node state is analyzed by graph convolution, the potential pollution path is identified, and a topological channel network is constructed.

The topological channel update network and the hydrological channel update network are merged and activated, and the joint attention is fused based on the initial adjacency matrix to obtain the dynamic adjacency matrix of each node.

Specifically, a dual-channel update network containing a hydrological channel network and a topological channel network is constructed; and a dynamic adjacency matrix reflecting a real-time system state is generated in combination with the joint attention and on the basis of the initial adjacency matrix, thereby providing a more accurate graph structure representation for prediction and control tasks in complex environments. That is, based on real-time hydrological data, the dynamic update of the connection weight between the nodes is driven, and the unsteady-state transport process of pollutants as hydrological conditions change is simulated.

The mask matrix includes the space mask matrix and the time mask matrix. The space mask matrix is used to mask the countercurrent relationship in the initial adjacency matrix. The time mask matrix is used to mask the future moment data in the initial adjacency matrix and data that exceeds a permitted upload delay.

Specifically, the causal attention mask is designed as follows:
through an upper triangular mask matrix

$${M}_{space}^{ij} ,$$

the unidirectionality of the water flow is enforced to avoid the propagation of countercurrent pollution, and a spatial constraint mask is constructed:

$${M}_{space}^{ij} = \{ \begin{matrix}1 , & node j is located upstream of i \\ 0 , & others\end{matrix} ;$$

in which:

$${M}_{space}^{ij}$$

represents the spatial mask matrix that only allows information flow from upstream to downstream.

As shown in FIG. 4, a strict causal convolutional kernel is used to ensure the causality, avoiding future data interference; and a time constraint mask is defined:

$${M}_{time}^{t , {t}^{′}} = \{ \begin{matrix}1 , & {t}^{′} \leq t \\ - ∞ , & {t}^{′} > t\end{matrix} ;$$

in which:

$${M}_{time}^{t , {t}^{′}}$$

represents a time mask matrix that only updates subsequent data with previous data.

A mask mechanism ensures that the model decision-making conforms to the law of water flow dynamics, and can constrain a search space, making the model training time shortened.

As an optional embodiment of the present invention, the dynamic adjacency matrix is expressed by a formula as:

Aₜ=A⁽⁰⁾⊙sigmoid(ΔA^{(hyd)}+ΔA^{(topo)})⊙Attention(Q,K,V);
in which, Aₜ represents the dynamic adjacency matrix; A⁽⁰⁾ represents the initial adjacency matrix; sigmoid represents an activation function for mapping an input variable between [0,1], and normalizing and adjusting feature weights, helping to stabilize training and keep the updated weights within a reasonable range;
ΔA^{(hyd)} represents the hydrological channel update network, and ΔA^{(hyd)}=f_{GNN} (Uₜ ⊗Vₜ);
ΔA^{(topo)} represents the topological channel update network, and ΔA^{(topo)}=f_{GNN} (hᵢ(t−1), Dₜ);
f_{GNN} represents the graph convolutional network; Uₜ represents the real-time flow velocity field matrix, reflecting a cross-sectional flow velocity of each node at time t; Vₜ represents the turbulent feature matrix (which may be expressed by a three-dimensional eddy viscosity coefficient, reflecting the mixing intensity of river turbulence); hᵢ(t−1) represents the hidden state at the previous moment; and Dₜ represents the degree matrix containing a real-time in/out degree of the nodes; and

$$Attention ( Q , K , V ) = Soft max ( \frac{{QK}^{T}}{\sqrt{{d}_{k}}} + {M}_{space} + {M}_{time} ) V ;$$

in which Q, K, V represent a query matrix, a key matrix and a value matrix respectively, which are respectively obtained by linear transformation through the hidden state at the previous moment; dₖ represents a vector dimension of the key matrix; M_{space} represents the space mask matrix; Mₜᵢₘₑ represents the time mask matrix; and Softmax represents a normalized exponential function which transforms the elements in an input vector into a probability distribution.

Specifically, Q represents the query matrix, in which the hidden state of the node is input as a query vector; K represents the key matrix, reflecting a historical state of each node or the features of its dependent adjacent nodes; V represents the value matrix, which contains a node feature (hidden state) that can be passed to a target site; dₖ represents a dimension of a key vector, which is used to scale a dot product result; Softmax represents a normalized exponential function, in which an attention score is converted into a probability distribution; Q (query matrix) indicates what information needs to be paid attention to at the current node i, and is a query vector obtained from the hidden state of the node i after W_{Q} projection change; K (key matrix) indicates what information other nodes j can provide, and is a query vector obtained from the hidden state hⱼ(t) of the node j after W_{K} projection change; and V (value matrix) indicates specific feature information actually passed to the target site, and is a query vector obtained from the hidden state hⱼ(t) of the node j after Wy projection change. Q (query matrix), K (key matrix), and V (value matrix) are all indirectly derived from the hidden state generated in step 3, but the feature projection is realized through different linear changes. W_{Q}, W_{K} and W_{V} are weight matrices of Q, K and V respectively, and together with the foregoing W_{S}, belong to inherent tuning parameters.

In summary, the dynamic adjacency matrix is used to represent an effective propagation relationship between the nodes in the watershed graph structure at the current time under real-time hydrological drive and causal constraints. By introducing the topological channel, the hydrological channel and the joint attention mechanism at the same time, the edge computing apparatus can more accurately simulate an unsteady-state transport process of pollutants in a complex watershed environment, and provide a basis for subsequent prediction, alarm level determination and automatic sampler control for the data-scarce points.

In the present embodiment, the above-mentioned dynamic connection weight update, hidden state update and dynamic adjacency matrix generation processes are all completed at the edge computing apparatus side. After a data acquisition gateway sends the real-time hydrological data and water quality parameter time series data at the observation nodes to the edge computing apparatus, the edge computing apparatus executes the continuous time state update and dynamic adjustment of the graph structure based on the watershed graph structure, then performs edge-side local reasoning on the plurality of data-scarce points by using the updated hidden state and the dynamic adjacency matrix, outputs the predicted values for the water quality parameters and alarm levels within the future preset time domain, sends the alarm event information to the central server, and meanwhile, uploads prediction results, alarm records, device operation state information and model operation logs to the central server to support subsequent model evaluation, parameter update and edge-cloud collaboration operation.

As an optional embodiment of the present invention, the central server acquires measured values for water quality parameters and the feature matrix constructed based on the historical hydrological data, the historical water quality data and the hydrological connectivity data, inputs the hidden state and the dynamic adjacency matrix into the initial neural network model to output predicted values for the water quality parameters, constructs a loss function based on the predicted values and the measured values, and determines an optimal hyperparameter through a K-fold cross-validation mechanism to obtain the updated liquid graph neural network model.

As an optional embodiment of the present invention, the loss function is as follows:

$$ℒ = \frac{1}{N} {\sum }_{i = 1}^{N} {( {y}_{i} - {\hat{y}}_{i} )}^{2} + {λ}_{1} Tr ( {Y}^{T} LY ) ;$$

in which, [symbol] represents a total loss function, which is an indicator used to measure a prediction error and a regularization effect; N represents a set of the nodes adjacent to the node i at the current moment; ŷᵢ represents the predicted value for the water quality parameter; y; represents a measured value for the water quality parameter; λ₁ represents the optimal hyperparameter; L represents a graph Laplacian matrix, which is used to quantify the structural information between nodes in the graph and constrain the spatial smoothness of predicted values of adjacent nodes; Y represents a matrix composed of the predicted values of the water quality parameters of the nodes; Tr represents a sum of all elements on the diagonals of the matrix; and λ₁Tr(Y^{T}LY) represents a graph Laplacian regular term.

In some embodiments, the central server is configured to perform training, verification, version update and issuance on the liquid graph neural network model. Specifically, the central server acquires training samples based on the historical hydrological data, the historical water quality data and the hydrological connectivity data, outputs the predicted values for the water quality parameters by using the initial liquid graph neural network model, and constructs a loss function based on the predicted values and the measured values to train and verify the model. Preferably, the K-fold cross-validation mechanism may be used for hyperparameter selection, and a Nash-Sutcliffe efficiency coefficient (NSE) may be used to evaluate the performances of the model.

Further, when hydrological seasonal features of the watershed change or a newly added monitoring node is introduced, the central server re-optimizes the model parameters and generates an incremental update packet. Seasonal changes may include changeover in a high flow period, a normal water period and a drought period, changes in rainfall and runoff conditions, and changes in confluence relationships caused by snow melting or freezing and thawing processes; the newly added nodes may include newly added hydrological monitoring nodes, newly added water quality monitoring nodes, or changes in sampling frequencies and acquisition parameters of the existing nodes. Since the above changes will cause changes in node feature distribution, edge weight relationship and local topology structure, the central server may re-construct training sample based on the updated hydrological connectivity data and the newly added node data, and optimize a dynamic connection weight calculation relationship, hidden state update parameters and a dynamic adjacency matrix generation strategy to generate the incremental update packet.

In some embodiments, the central server issues a model update instruction and the incremental update packet to the edge computing apparatus only when the network connection state satisfies a preset stable condition. The preset stable condition may include at least one or a combination thereof of a communication link remaining continuously normal, a packet loss rate falling below a preset stabilization threshold, and a communication delay falling below a preset stabilization threshold. Preferably, a stable packet loss rate threshold may be set to 1% to 10%, a stable delay threshold may be set to 0.5 s to 5 s, and a stable duration may be set to 30 s to 10 min.

As shown in FIG. 6, after receiving the historical hydrological data, the historical water quality data and the hydrological connectivity data, the central server completes training sample construction, model training, verification, hyperparameter determination and incremental update packet generation, and issused an updated model version, model parameters or strategy configuration to the edge computing apparatus under a stable condition of the network to support subsequent edge-side local reasoning.

In some embodiments, the edge computing apparatus is configured to call the liquid graph neural network model issued by the central server, perform local reasoning on the plurality of data-scarce points at the edge side, and output predicted values for water quality parameters and corresponding alarm levels at the plurality of data-scarce points within the future preset time domain; and when the predicted value for the water quality parameter exceeds a preset threshold or the alarm level reaches a preset level, generate alarm event information and send the alarm event information to the central server, and meanwhile send the hardware action instruction to upstream nodes or downstream nodes associated with the data-scarce points, to control their automatic samplers to perform encrypted sampling.

Specifically, in the present embodiment, the edge computing apparatus receives real-time hydrological data of each node from the data acquisition gateway and water quality parameter time series data from the observation nodes, and after the updating of the aforementioned watershed graph structure, dynamic connection weight, hidden state and dynamic adjacency matrix is completed, predicts the water quality parameters at the plurality of data-scarce points within the future preset time domain by using the locally stored liquid graph neural network model. The preset time domain may be set to a period of several hours in the future, one day in the future or seven days in the future according to specific application requirements. The edge computing apparatus then generates the corresponding alarm level according to the predicted value and performs event determination according to the preset threshold value or the preset level. When an early warning condition is met, the edge computing apparatus generates alarm event information and sends the alarm event information to the central server, and at the same time outputs local alarm information at the edge side, to control its automatic sampler to perform encrypted sampling.

In some embodiments, the alarm level may be determined according to at least one of an out-of-limit degree, a duration, a change rate and a multi-index combination risk among the predicted values for the water quality parameters relative to a corresponding pollutant threshold. The alarm levels may be divided into no alarm, low-level alarm, intermediate-level alarm and high-level alarm, or divided into first, second and third alarm levels.

In some embodiments, after receiving the model update instruction and the incremental update packet issued by the central server, the edge computing apparatus may perform integrity check and version consistency check; when the check passes, updates a version of the local liquid graph neural network model and calls the updated model to perform edge-side local reasoning; and when the check fails, continues to run the current model version. Preferably, the edge computing apparatus may complete the model update by background loading and hot switching to avoid the interruption of prediction and early warning.

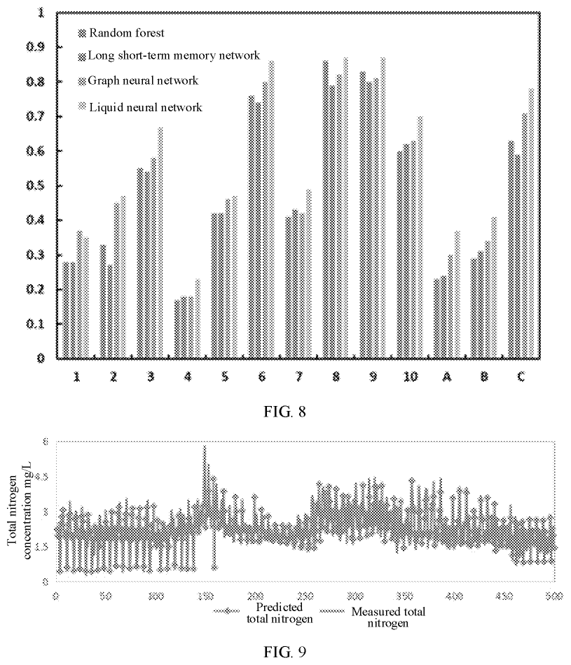

As shown in FIG. 7, FIG. 7 shows a schematic diagram of edge reasoning and local disaster tolerance in an embodiment of the present invention. Under normal conditions, the edge computing apparatus receives a data stream sent by the data acquisition gateway, and calls the model sent by the central server to perform local reasoning and alarm output. As shown in FIG. 8 and FIG. 9, FIG. 8 shows a comparison diagram of predicted NSE effects of different models, and FIG. 9 shows a comparison diagram between prediction and actual measurement of total nitrogen in water quality parameters. Compared with models such as random forest, long short-term memory network and common graph neural network, it can be seen that the liquid graph neural network scheme adopted in the present embodiment has higher prediction accuracy and more stable prediction performance, and the total nitrogen prediction result can also better follow the measured change trend, indicating that it is suitable for prediction and early warning for the data-scarce points under a monitoring-scarce scenario.

In some embodiments, the network being interrupted or the response delay of the central server reaching the preset condition includes at least one of interruption of a communication link between the edge computing apparatus and the central server, a packet loss rate exceeding a preset threshold, and a communication delay exceeding a preset delay threshold. Preferably, the packet loss rate may be calculated according to a preset sliding time window, which may be 1 min, 5 min or 10 min; and a packet loss rate threshold may be set to 5% to 30%, a communication delay threshold may be set to 1 s to 30 s, and a communication link interruption duration threshold may be set to 10 s to 10 min. Further, a communication abnormality condition is determined to be reached when the link interruption duration exceeds a preset duration, or the packet loss rate exceeds a threshold for N consecutive detection cycles within the sliding time window, or the communication delay exceeds a threshold for M consecutive detection cycles, wherein N and M may be set to 2 to 10, preferably 3 to 5.

When the network is interrupted or the response delay of the central server reaches the preset condition, the edge computing apparatus calls a locally stored liquid graph neural network model version and locally cached real-time hydrological data, and continues to execute edge reasoning, alarm level determination and local alarm output for the plurality of data-scarce points, to maintain the local continuous early warning capability.

In some embodiments, when the communication link restores, the packet loss rate is lower than a preset restoration threshold, and the communication delay is lower than a preset restoration threshold and lasts for a preset restoration duration, the edge computing apparatus switches back to an edge-cloud collaboration operation mode from a local independent operation mode. Preferably, a packet loss rate restoration threshold may be set to 1% to 10%, a delay restoration threshold may be set to 0.5 s to 5 s, and a restoration duration may be set to 30 s to 10 min. Further, in order to prevent frequent switching due to fluctuations of a network state near critical conditions, an abnormal trigger threshold for entering the local independent operation mode and a restoration threshold for restoring to the edge-cloud collaboration operation mode may be set to different values to form a hysteresis switching mechanism.

As shown in FIG. 7, under abnormal conditions, the edge computing apparatus can continuously perform prediction, alarm level determination and alarm output based on the local model and the locally cached data, thereby ensuring that the prediction and early warning for the water quality at the data-scarce points are not interrupted, and improving the reliability and emergency decision-making support capability of the system under complex conditions.

In some embodiments, the edge computing apparatus and the central server operate in an edge-cloud collaboration mode. Specifically, the edge computing apparatus uploads the prediction results, alarm records, device operation state information and model operation logs at the data-scarce points to the central server; the central server executes model evaluation, policy adjustment and version synchronization according to information uploaded by the edge side, and generates a model update strategy, model parameters or an incremental update packet related to prediction and early warning for the data-scarce points; and the edge computing apparatus updates an edge-side reasoning process according to the model update strategy, the model parameters or the incremental update packet issued by the central server, so as to complete edge-side model collaboration update under the condition of not interrupting the prediction and early warning output at the data-scarce points.

Further, when the edge computing apparatus restores from the local independent operation mode to the edge-cloud collaboration operation mode, the prediction results, the alarm records, the device state information and model operation logs generated during the local independent operation may also be supplemented to the central server, such that the central server can complete historical data archiving, event supplement registration, model evaluation correction and operation state synchronization.

The system of the present invention is configured to perform specific industrial applications in a real watershed environment, including but not limited to: (1) automatically triggering an upstream or downstream automatic sampler to perform encrypted sampling based on the prediction results; (2) generating a control instruction and driving field devices to implement pollution response measures when water quality parameters exceed the threshold; (3) continuously outputting early warning information through an edge-side local model in the event of network interruption; and (4) providing real-time decision-making support data for watershed regulators to reduce environmental risks.

The liquid graph neural network is not only used for data analysis, but interacts with a physical hydrological system through the edge computing apparatus to directly convert the prediction result into a control signal, thereby achieving monitoring and intervention on the actual water environment.

The foregoing is only a specific implementation of the present disclosure, such that those skilled in the art can understand or implement the present invention. A variety of modifications of these embodiments will be obvious to those skilled in the art, and the general principles defined herein may be implemented in other embodiments without departing from the spirit or scope of the present invention. Accordingly, the present invention will not be limited to these embodiments shown herein, but will conform to the widest range consistent with the principles and novel features disclosed herein.

1-10. (canceled)

11. A prediction and early warning system for the water quality at a monitoring-scarce point based on a liquid graph neural network, wherein a prediction result output by the system is used to directly control a physical sampling device or an early warning device, thereby constituting a technical intervention to an actual water environment;
the system comprising:
an edge computing apparatus that comprises a processor, a communication module and an I/O control module, wherein the edge computing apparatus is configured to convert the prediction result into a control signal and drive an automatic sampler, an electromagnetic valve or an alarm device to execute physical operations; the system constitutes real-time monitoring and intervention for an actual watershed water environment;
watershed front-end monitoring devices that are deployed at a plurality of monitoring nodes, each watershed front-end monitoring device comprising a hydrological sensor and a communication module, and the each watershed front-end monitoring device deployed at an observation node further comprising a water quality sensor and an automatic sampler, wherein the monitoring nodes are divided into observation nodes deployed with the water quality sensors and data-scarce points where no water quality sensor is deployed; and
a central server configured to perform training, verifying, version update and event data synchronization on the liquid graph neural network model, wherein when a network is interrupted or a response delay of the central server reaches a preset condition, the edge computing apparatus calls a locally stored liquid graph neural network model version and locally cached real-time hydrological data, and continues to execute edge reasoning and early warning output for the plurality of data-scarce points
wherein the edge computing apparatus is in communication connection with the watershed front-end monitoring devices, the edge computing apparatus comprises a memory and the processor, the memory stores a directed initial watershed graph, an initial adjacency matrix, a degree matrix, a feature matrix, a liquid graph neural network model, a physical constraint rule and a computer program therein, and the processor performs the following steps:
receiving real-time hydrological data and real-time water quality data collected by the observation nodes, and performing error value deletion, threshold capping, missing value filling, normalization and time step alignment processing on the real-time hydrological data and the real-time water quality data to form an input data set;
calling the pre-stored directed initial watershed graph, and determining an initial connection weight between every two downstream adjacent nodes according to a riverway distance, historical flow velocities, a riverbed permeability coefficient and hydrological connectivity data, and then determining a dynamic correction factor according to real-time velocity gradients and rainfall changes to dynamically correct edge weights in the directed initial watershed graph to generate a dynamic watershed graph; and
inputting the feature matrix, the dynamic watershed graph and the dynamic connection weight into the liquid graph neural network model, and generating a dynamic adjacency matrix by using hidden state update driven by a preset liquid differential equation, a topological channel update network constructed based on the hidden state at the previous moment and a hydrological channel update network as well as a joint attention fused with a space mask matrix and a time mask matrix, and accordingly outputting predicted values for water quality parameters and corresponding alarm levels at the plurality of data-scarce points within a future preset time domain;
when the predicted value for the water quality parameter exceeds a preset threshold or the alarm level reaches a preset level, generating an alarm data packet containing a hardware action instruction, determining an upstream node or a downstream node associated with the data-scarce point that reaches an alarm condition according to the dynamic adjacency matrix, and sending the hardware action instruction to the determined node to control its automatic sampler to perform encrypted sampling.

12. The prediction and early warning system for the water quality at a monitoring-scarce point based on the liquid graph neural network according to claim 11, wherein constructing the directed initial watershed graph comprises:
acquiring river data, historical hydrological data and hydrological connectivity data of each monitoring node;
determining the initial connection weight between each pair of adjacent nodes according to a riverway distance, historical flow velocities and a riverbed permeability coefficient between each pair of adjacent nodes having a downstream relationship, and establishing directed edges only when there is a downstream relationship and hydrological connection;
constructing the initial adjacency matrix according to the riverway distance, an average node distance between the nodes and a distance attenuation coefficient, and constructing the degree matrix according to the initial adjacency matrix; and
constructing the feature matrix of each node according to the historical hydrological data and the hydrological connectivity data of the node and historical water quality data of the observation nodes, wherein the feature matrix at least comprises static attributes, dynamic time series features, spatial topological features and water quality characterization features at the data-scarce points estimated according to historical water quality data of adjacent observation nodes,
wherein the water quality characterization features at the data-scarce points are estimated according to the hydrological connectivity data and the historical water quality data of adjacent observation nodes.

13. The prediction and early warning system for the water quality at a monitoring-scarce point based on a liquid graph neural network according to claim 11, wherein dynamically updating the dynamic connection weight between the nodes comprises:
determining a dynamic correction factor between each pair of adjacent nodes according to the initial connection weight between each pair of adjacent nodes and the real-time hydrological data of each node, wherein the dynamic correction factor is used to characterize a physical driving effect of a linear combination of the flow velocity gradients and rainfall changes on a pollutant diffusion rate; and
determining the dynamic connection weight between each pair of adjacent nodes according to the initial connection weight and the dynamic correction factor, wherein
the dynamic correction factor is used to characterize physical driving effects of the flow velocity gradients and rainfall changes on the pollutant diffusion rate.

14. The prediction and early warning system for the water quality at a monitoring-scarce point based on a liquid graph neural network according to claim 13, wherein the hidden state update in the liquid graph neural network model is driven by a preset liquid differential equation and is used to simulate continuous evolutions of pollutants in time and space, comprising:
determining a feature vector of the feature matrix of each node; and generating a comprehensive input of each node in the preset liquid differential equation according to the feature vector, the dynamic connection weight and a set of nodes adjacent to said node, so as to output the hidden state of each node, wherein
the preset liquid differential equation is:
$$\frac{{dh}_{i} ( t )}{dt} = - {h}_{i} ( t ) + σ ( I ( t ) + b ) ;$$
$$I ( t ) = {W}_{s} [ {x}_{i} ( t ) ] ⊕ {\sum }_{j ∈ 𝒩 ( i )} {α}_{ij} ( t ) {h}_{j} ( t - Δ t ) ;$$
in which, hᵢ(t) represents a hidden state of a node i at time t; σ represents a nonlinear activation function; b represents a learnable bias term; Wₛ represents a learnable parameter matrix; [xᵢ(t)] represents the feature vector of the node i at time t; αᵢⱼ(t) represents the dynamic connection weight; ⊕ represents a feature splicing operation; Δt represents a time delay; I(t) represents the comprehensive input; j represents the node adjacent to the node i; and N(i) represents a set of the nodes adjacent to the node i.

15. The prediction and early warning system for the water quality at a monitoring-scarce point based on a liquid graph neural network according to claim 14, wherein generating the dynamic adjacency matrix comprises:
determining an attention score based on the hidden state of each node at the previous moment; fusing the space mask matrix and the time mask matrix to obtain the joint attention;
constructing the topological channel update network according to the hidden state of each node at the previous moment and the degree matrix, and constructing the hydrological channel update network according to a real-time flow velocity field matrix and a turbulent feature matrix corresponding to the real-time hydrological data; and
merging and activating the topological channel update network and the hydrological channel update network, and obtaining the dynamic adjacency matrix of each node based on the initial adjacency matrix and by fusing the joint attention, wherein the dynamic adjacent matrix is used to determine an upstream node or a downstream node corresponding to the data-scarce point that reaches an alarm condition, to control its automatic sampler to perform encrypted sampling, wherein
the space mask matrix is used to mask edges that do not meet a downstream connection relationship; and the time mask matrix is used to mask future moment data and data that exceeds a permitted upload delay; and
the dynamic adjacent matrix is used to determine an upstream node or a downstream node corresponding to the data-scarce point that reaches the alarm condition, to control its automatic sampler to perform encrypted sampling.

16. The prediction and early warning system for the water quality at a monitoring-scarce point based on a liquid graph neural network according to claim 15, wherein the dynamic adjacency matrix is jointly determined by the initial adjacency matrix, the hydrological channel update network, the topological channel update network and the joint attention, and the joint attention is generated by a query matrix, a key matrix and a value matrix as well as the space mask matrix and the time mask matrix:
Aₜ=A⁽⁰⁾⊙sigmoid(ΔA^{(hyd)}+ΔA^{(topo)})⊙Attention(Q,K,V);
in which, Aₜ represents the dynamic adjacency matrix; A⁽⁰⁾ represents the initial adjacency matrix; sigmoid represents an activation function;
ΔA^{(hyd)} represents the hydrological channel update network, and ΔA^{(hyd)}=f_{GNN} (Uₜ⊗Vₜ);
ΔA^{(topo)} represents the topological channel update network, and ΔA^{(topo)}=f_{GNN} (hᵢ(t−1),Dₜ);
f_{GNN} represents a graph convolutional network; Uₜ represents a real-time flow velocity field matrix; Vₜ represents a turbulent feature matrix; hᵢ(t−1) represents the hidden state at the previous moment; Dₜ represents the degree matrix;
$$Attention ( Q , K , V ) = Soft max ( \frac{{QK}^{T}}{\sqrt{{d}_{k}}} + {M}_{space} + {M}_{time} ) V ;$$
in which, Q,K,V represent a query matrix, a key matrix and a value matrix respectively, which are respectively obtained by linear transformation through the hidden state at the previous moment; dₖ represents a vector dimension of the key matrix; M_{space} represents the space mask matrix; Mₜᵢₘₑ represents the time mask matrix; and Softmax represents a normalized exponential function, wherein the time mask matrix is used to shield future moment data and invalid data packets that exceed the permitted upload delay, and the space mask matrix is used to shield countercurrent edges that do not meet the downstream connection relationship.

17. The prediction and early warning system for the water quality at a monitoring-scarce point based on a liquid graph neural network according to claim 11, wherein the central server is configured to perform training, verification, version update and issuance on the liquid graph neural network model, and to perform training, verification, version update and issuance on the liquid graph neural network model comprises:
the central server acquires training samples constructed based on the historical hydrological data, the historical water quality data and the hydrological connectivity data; trains and verifies the liquid graph neural network model according to the training samples to obtain an updated liquid graph neural network model; when hydrological seasonal features of the watershed change or monitoring node data is newly added, re-optimizes model parameters to generate an incremental update packet; and when a network connection state meets a preset stable condition, issues a model update instruction and the incremental update packet to the edge computing apparatus to achieve edge-side model version update without interrupting the prediction and early warning output for the data-scarce points.

18. The prediction and early warning system for the water quality at a monitoring-scarce point based on a liquid graph neural network according to claim 11, wherein
the edge computing apparatus is configured to call the liquid graph neural network model issued by the central server, perform local reasoning on the plurality of data-scarce points at the edge side, and output predicted values for water quality parameters and corresponding alarm levels at the plurality of data-scarce points within a future preset time domain; and
when the predicted value for the water quality parameter exceeds a preset threshold or the alarm level reaches a preset level, generate alarm event information and send the alarm event information to the central server, and meanwhile send the hardware action instruction to upstream nodes or downstream nodes associated with the data-scarce points according to the dynamic adjacency matrix, to control their automatic samplers to perform encrypted sampling.

19. The prediction and early warning system for the water quality at a monitoring-scarce point based on a liquid graph neural network according to claim 11, wherein
the network being interrupted or the response delay of the central server reaching the preset condition includes at least one of interruption of a communication link between the edge computing apparatus and the central server, a packet loss rate exceeding a preset threshold, and a communication delay exceeding a preset delay threshold; and
when the network is interrupted or the response delay of the central server reaches the preset condition, the edge computing apparatus calls a locally stored liquid graph neural network model version and locally cached real-time hydrological data, and continues to execute edge reasoning, alarm level determination and local early warning output for the plurality of data-scarce points.

20. The prediction and early warning system for the water quality at a monitoring-scarce point based on a liquid graph neural network according to claim 11, wherein the edge computing apparatus and the central server operate in an edge-cloud collaboration mode, comprising:
the edge computing apparatus uploads the prediction results, alarm records, device operation state information and model operation logs at the data-scarce points to the central server;
the central server executes model evaluation, policy adjustment and version synchronization according to information uploaded by the edge side, and generates a model update strategy, model parameters or an incremental update packet related to prediction and early warning for the data-scarce points; and
the edge computing apparatus updates an edge-side reasoning process according to the model update strategy, the model parameters or the incremental update packet issued by the central server, so as to complete edge-side model collaboration update under the condition of not interrupting the prediction and early warning output at the data-scarce points.
